(19) United States
(12) Patent Application Publication
(10) Pub. No.: US 2026/0174733 A1
(43) Pub. Date: Jun. 25, 2026

(54) PROLIPOSOMAL DELIVERY SYSTEM AND METHOD FOR PREPARING THE SAME

(71) Applicant: BOTANIC HEALTHCARE PVT. LTD., Hyderabad (IN)

(72) Inventors: GAURAV SONI, Hyderabad (IN); HEBBANI NAGARAJAPPA SHIVAPRASAD, Hyderabad (IN); GARAGA MACHIRAJU, Hyderabad (IN); ARNAB CHATTERJEE, Hyderabad (IN); SRAVANI THIMMANNAGARI, Hyderabad (IN); DIVANJI MANOHAR SHRIRANGA, Hyderabad (IN); MANOJ B GOMKALE, Hyderabad (IN); SIDDARTHA MAJI, Hyderabad (IN); GANDAM LOHITHA, Hyderabad (IN); CHERUPALLI SREE RAMYA, Hyderabad (IN)

(21) Appl. No.: 19/425,068

(22) Filed: Dec. 18, 2025

(30) Foreign Application Priority Data
Dec. 20, 2024 (IN) 202441101549

(51) Int. Cl. A61K 31/375; A61K 9/107; A61K 9/1277; A61K 47/24; A61K 47/26; A61K 47/36

(52) U.S. Cl. CPC A61K 31/375; A61K 9/1075; A61K 9/1277; A61K 47/24; A61K 47/26; A61K 47/36

(57) ABSTRACT

A proliposomal delivery system and method for preparing the same is provided. The proliposomal delivery system involves a proliposomal formulation incorporating liposomal entrapment of vitamin C within a nano-emulsion that is stabilized using partially hydrolyzed guar gum. The proliposomal formulation includes ascorbic acid as the active ingredient, sunflower lecithin with a phosphatidylcholine content as an encapsulating material, and partially hydrolyzed guar gum as a dietary polysaccharide. The proliposomal formulation is a safer and more bioavailable form of vitamin C designed to minimize gastric discomfort. The proliposomal formulation offers a nutraceutical supplement aimed at managing oxidative stress, enhancing immune system function, supporting heart health, and improving skin. The process provided for preparing the proliposomal formulation is simple, cost-effective and scalable.

### CROSS-REFERENCE TO RELATED APPLICATIONS

This application claims the benefit of Indian Patent Application No. IN 202441101549, filed on Dec. 20, 2024, the content of which is incorporated herein by reference in its entirety.

### FIELD OF INVENTION

The present disclosure relates generally to the field of nutraceuticals formulations and delivery systems. In particular, the present disclosure pertains to a proliposomal delivery system and method for preparing the same.

### BACKGROUND

Vitamin C, also known as ascorbic acid, is an essential nutrient with critical roles in various physiological processes, including collagen synthesis, immune function, and antioxidant defence. Vitamin C is widely used as a dietary supplement to combat oxidative stress, enhance immunity, and promote skin and cardiovascular health. However, traditional vitamin C formulations often face significant limitations in terms of stability, bioavailability, and gastrointestinal tolerability. These challenges hinder the effectiveness of the nutrient, particularly in individuals requiring higher doses for therapeutic purposes.

Major problem with conventional vitamin C supplements is their limited bioavailability. Ascorbic acid is water-soluble and prone to rapid degradation in the gastrointestinal tract, leading to poor absorption and reduced systemic availability. Research has shown that only a small fraction of orally administered vitamin C is absorbed into the bloodstream, with the majority being excreted in urine. This inefficiency necessitates higher doses, which further aggravates other issues, such as gastric irritation.

Another significant issue is the instability of ascorbic acid. Ascorbic acid is highly susceptible to oxidative degradation when exposed to environmental factors such as light, heat, and air. This instability reduces the efficacy of vitamin C supplements over time and poses challenges in storage and transportation. Efforts to address this issue, such as using buffered or time-release formulations, have shown limited success and often fail to provide consistent results.

Gastrointestinal discomfort is a common side effect associated with high-dose vitamin C supplementation. Ascorbic acid's acidic nature can irritate the stomach lining, leading to symptoms such as nausea, diarrhoea, and abdominal pain. This limits the tolerability of higher doses, which are often required to achieve therapeutic effects, particularly in cases of oxidative stress, immune suppression, or skin conditions. Microencapsulation and other drug delivery systems have shown promise although the potentiality of gastric discomfort due to higher loading was unavoidable.

Therefore, there is a need for an improved vitamin C delivery system that overcomes the limitations of conventional formulations.

### OBJECTS OF THE PRESENT DISCLOSURE

Some of the objects of the present disclosure, which at least one embodiment herein satisfies are as listed herein below.

An object of the present invention is to provide a proliposomal vitamin C formulation with enhanced bioavailability.

Another object of the present invention is to develop a stable vitamin C delivery system that resists oxidative degradation.

Another object of the present invention is to provide a method for preparing the proliposomal vitamin C formulation.

### SUMMARY

This summary is provided to introduce a selection of concepts in a simplified form that is further described below in the detailed description section. This summary is not intended to identify key features or essential features of the claimed subject matter, nor is it intended to be used as an aid in determining the scope of the claimed subject matter.

Aspects of the present disclosure relates to nutraceutical formulations, more specifically to advanced vitamin delivery systems designed to enhance bioavailability and stability. The formulation is suitable for use in health supplements, functional foods, and preventive healthcare applications aimed at providing antioxidant and cardio protective benefits.

In an aspect, the present disclosure provides a proliposomal formulation for vitamin C delivery incorporating a dietary fiber coating to improve gastric tolerance, cellular uptake, and absorption. The proliposomal formulation includes 70 to 75 wt % ascorbic acid (vitamin C) as the active ingredient, encapsulated within liposomes formed using 4 to 5 wt % of sunflower lecithin rich in 50 to 70% phosphatidylcholine more preferably with 70% phosphatidylcholine content. The formulation also includes 3 to 5 wt % partially hydrolyzed guar gum as a dietary fiber coating. The formulation also includes 14 to 20 wt % of a polysaccharide carrier and 1 to 2 wt % of surfactant preferably non-ionic. The dietary polysaccharide coating stabilizes the liposomal structure, enhances gastric tolerance, and improves cellular uptake of vitamin C. The formulation exhibits significantly enhanced bioavailability compared to conventional vitamin C supplements.

In another aspect, the present disclosure provides a method for preparing a proliposomal formulation, comprising steps of:
a) mixing 4 to 5 wt % of sunflower lecithin with ethanol at a temperature of 50 to 55° C. under controlled stirring to obtain an organic phase;
b) mixing the organic phase of step a) with an aqueous phase containing 1 to 2 wt % of surfactant preferably non-ionic and mainly polysorbates with high HLB value to form a stable emulsion followed by cooling the emulsion to stabilize vesicles;
c) incorporating 70 to 75 wt % of ascorbic acid into the cooled stable emulsion of step b) with continuous stirring for 30 to 45 minutes to form a homogeneous emulsion;
d) adding 3 to 5 wt % of a dietary fiber solution to the homogeneous emulsion of step c) to create a final liposomal emulsion with loaded ascorbic acid;
e) homogenizing the final liposomal emulsion of step d) at pressure in a range of 200-300 bar to achieve vesicle size ranging from 100 to 200 nm for a homogenised nano-emulsion; and
f) spray-drying the homogenized nano-emulsion of step e) with 14 to 20 wt % of a polysaccharide carrier solution to produce a proliposomal formulation.

Various objects, features, aspects and advantages of the inventive subject matter will become more apparent from the following detailed description of preferred embodiments.

### BRIEF DESCRIPTION OF DRAWINGS

The accompanying drawings are included to provide a further understanding of the present disclosure and are incorporated in and constitute a part of this specification. The drawings illustrate exemplary embodiments of the present disclosure and, together with the description, serve to explain the principles of the present disclosure.

FIG. 1 illustrates steps involved in a method for preparing a proliposomal formulation, in accordance with an embodiment of the present disclosure.

FIG. 2 illustrates mean concentration of Plasma Vitamin C with different formulations, in accordance with an embodiment of the present disclosure.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present invention.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, numbers have been used for quantifying weight percentages, ratios, and so forth, to describe and claim certain embodiments of the invention and are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Various terms as used herein are shown below. To the extent a term used in a claim is not defined below, it should be given the broadest definition persons in the pertinent art have given that term as reflected in printed publications and issued patents at the time of filing.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

The description that follows, and the embodiments described therein, is provided by way of illustration of an example, or examples, of particular embodiments of the principles and aspects of the present invention. These examples are provided for the purposes of explanation, and not of limitation, of those principles and of the disclosure.

It should also be appreciated that the present invention can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements a, b, and c, and a second embodiment comprises elements b and d, then the inventive subject matter is also considered to include other remaining combinations of a, b, c, or d, even if not explicitly disclosed.

The terms “composition” or “formulation” or “proliposomal formulation” or “proliposomal vitamin C formulation” are used herein interchangeably with same meaning throughout the specification.

Aspects of the present disclosure relates to nutraceutical formulations, more specifically to advanced vitamin delivery systems designed to enhance bioavailability and stability. The disclosure provides a proliposomal formulation for vitamin C delivery. The disclosure addresses challenges in conventional vitamin C supplements, such as low bioavailability and gastric discomfort, and is applicable to the management of oxidative stress, immune system support, cardiovascular health, and skin improvement.

Accordingly, in an aspect, the present disclosure provides a proliposomal formulation, including 70 to 75 wt % ascorbic acid as the active ingredient, 4 to 5 wt % of sunflower lecithin with phosphatidylcholine content in the range of 50-70% more preferably 70% as an encapsulating material, and 3 to 5 wt % of partially hydrolyzed guar gum as dietary fiber. The formulation also includes 14 to 20 wt % of a polysaccharide carrier and 1 to 2 wt % of surfactant. The formulation stabilizes liposomal structure and improves bioavailability of the active ingredient, making the formulation an ideal nutraceutical supplement. The formulation is configured to enhance gastric tolerance and cellular uptake of vitamin C.

In one embodiment, the formulation includes vitamin C (ascorbic acid) not less than 70 wt %, the sunflower lecithin of phosphatidylcholine content 50% not less than 4 wt %, polysorbate 80 not less than 1 wt %, and the partially hydrolysed guar gum not less than 5 wt % and the polysaccharide carrier not less than 20 wt %.

In one embodiment, the formulation includes vitamin C (ascorbic acid) not less than 75 wt %, the sunflower lecithin of phosphatidylcholine content 70% not less than 5 wt %, polysorbate 80 not less than 2 wt %, and the partially hydrolysed guar gum not less than 3.5 wt % and the polysaccharide carrier not less than 14.5 wt %.

In various embodiments, the surfactant is selected from a group of hydrophilic emulsifiers consisting of polysorbate 80, polysorbate 60, polysorbate 40. Polysorbate 80 is a non-ionic surfactant with a high hydrophilic-lipophilic balance (HLB), and effectively reduces the interfacial tension between the lipid (organic) and aqueous phases, enabling the formation of a stable and uniform water-in-oil emulsion. With a known history in liposomes to offer enhanced drug solubility and loading efficiency and also being a surfactant, it aids to prevent vesicle aggregation and supports the encapsulation and stabilization of the water-soluble vitamin C (ascorbic acid) within the lipid bilayer.

In certain embodiments, the sunflower lecithin comprises a phosphatidylcholine content ranging from 50 to 70%. The sunflower lecithin is derived from sunflower oil and contains a phosphatidylcholine concentration optimized for liposomal encapsulation.

In various embodiments, the dietary fibers are selected from a group of soluble dietary fibres with clinical evidence on gut health improvement consisting of partially hydrolyzed guar gum, guar gum, pectin, and a combination thereof.

In certain embodiments, the polysaccharide carrier is selected from a group consisting of maltodextrin, gum acacia and a combination thereof.

In certain embodiments, the proliposomal formulation comprises liposomal vesicles with a diameter ranging from 100 to 200 nm and a solid content of 25 to 27%.

In various embodiments, the formulation involves liposomal entrapment of vitamin C within a nano-emulsion that is stabilized using partially hydrolyzed guar gum. Stabilization of the nano-emulsion employs Dietary Fiber Stabilized (DFS) Technology to enhance gastric tolerance and improve cellular uptake, leading to significantly better absorption and bioavailability compared to conventional vitamin C formulations.

As used herein the term “Dietary Fiber Stabilized (DFS) Technology” refers to a method of utilizing dietary fibers to enhance the stability and functionality of bioactive compounds, particularly in nutraceutical and pharmaceutical formulations. In the present disclosure, DFS Technology involves the coating of dietary polysaccharide, such as partially hydrolyzed guar gum possessing cationic charges that can electrostatically stabilize nano-emulsion. The stabilized nano emulsion is expected to improve oral delivering characteristics of liposomal delivery systems reflecting enhanced gastric stability and better absorption of vitamin C.

In another aspect, the present disclosure provides a method for preparing a proliposomal formulation.

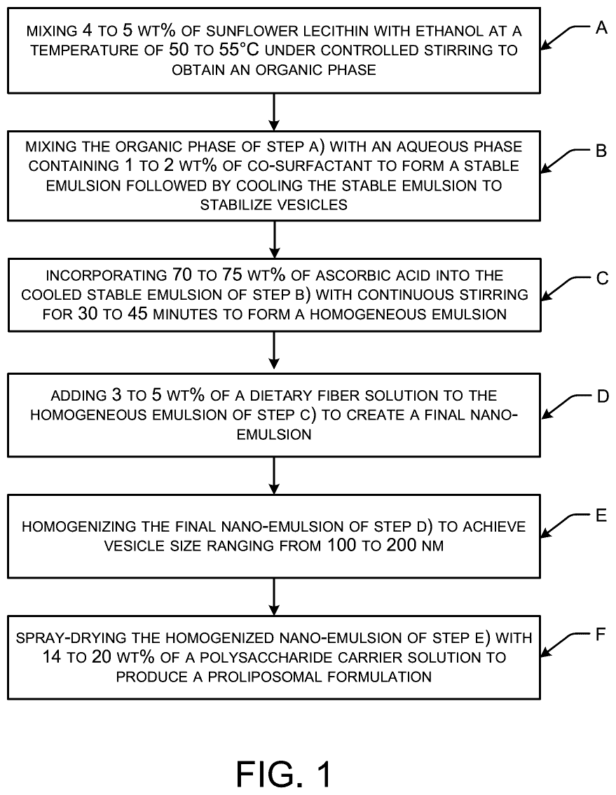

FIG. 1 shows steps involved in the method for preparing the proliposomal formulation, including steps of:
a) mixing 4 to 5 wt % of sunflower lecithin with ethanol at a temperature of 50 to 55° C. under controlled stirring to obtain an organic phase;
b) mixing the organic phase of step a) with an aqueous phase containing 1 to 2 wt % of surfactant preferably non-ionic and mainly polysorbates with high HLB value to form a stable emulsion followed by cooling the emulsion to stabilize vesicles;
c) incorporating 70 to 75 wt % of ascorbic acid into the cooled stable emulsion of step b) with continuous stirring for 30 to 45 minutes to form a homogeneous emulsion;
d) adding 3 to 5 wt % of a dietary fiber solution to the homogeneous emulsion of step c) to create a final liposomal emulsion with loaded ascorbic acid;
e) homogenizing the final liposomal emulsion of step d) at pressure in a range of 200-300 bar to achieve vesicle size ranging from 100 to 200 nm for a homogenised nano-emulsion;
f) spray-drying the homogenized nano-emulsion of step e) with 14 to 20 wt % of a polysaccharide carrier solution to produce a proliposomal formulation.

In various embodiments, the mixing at step b) is carried out by stirring vigorously for a duration of 20 to 30 minutes at 50 to 55° C. In one embodiment, the surfactant used is preferably non-ionic and mainly polysorbates with high HLB value, most preferably polysorbate 80.

In certain embodiments, the stable emulsion is cooled at step b) at a temperature ranging from 30 to 35° C. to stabilize vesicles.

In certain embodiments, the final nano-emulsion is homogenized at step e) using a high-pressure homogenizer. As used herein the term “high-pressure homogenizer” refers to a mechanical device used to create uniform and stable emulsions, suspensions, or dispersions by forcing a liquid or semi-solid mixture through a narrow gap at very high pressure. The process reduces the size of particles, droplets, or vesicles within the mixture, achieving a consistent and fine distribution.

In various embodiments, the spray-drying at step f) is carried out at an inlet temperature of 150 to 155° C. and an outlet temperature of 90 to 95° C. As used herein the term “spray-drying” refers to a process that turns a liquid or slurry into a dry powder by rapidly drying it with hot gas.

In an exemplary embodiment, the method also includes a pre-homogenisation solution of the emulsifier (organic phase) preparation with preferably sunflower lecithin with the phosphatidylcholine content of 70%, in an equal weight of a food grade organic solvent alcohol such as ethanol as a green solvent. This step is carried out in a closed reactor system equipped with a reflux condenser to prevent ethanol loss and ensure process efficiency. The emulsification is conducted at a controlled temperature of 50-55° C., preferably maintained at 50° C., to achieve a homogeneous and stable organic phase suitable for subsequent emulsion formation. Further the method involves addition of the organic phase to the aqueous phase of 10-20 volumes of water with the surfactant mainly polysorbate 80, preferably with a high HLB value for a stable water-in-oil emulsion in the above temperature range.

Furthermore, the method involves relieving solution temperature preferably in the ambient temperature range of 30-35° C. with cooling circulation in the reactor with a pulsated jerk and hold time of 3 to 4 hours to stabilise liposomal vesicles obtained in the process. Further, the method involves sequential loading of vitamin C in portions under high-speed stirring with additional volume of water of 1.5-2.0 times its weight, preferably 2.0 times the weight of ascorbic acid in water. The complete dissolution takes an average stirring time of 30-45 minutes, more preferably 45 minutes. Again, the vehicle loaded liposomal system was stabilised for a hold time of 2-3 hours for better stabilisation and better encapsulation. Further, with methodology of DFS (Dietary Fibre Stabilisation) to the above solution partially hydrolysed guar gum solution in water with 1.5-2 times hydrolysed guar gum's weight, more preferably 2 times its weight was added in portions with slow stirring to facilitate better layering and passed through high pressure homogenisation system at a pressure scale of 200-300 bar in order to achieve a narrow liposomal size of 100-200 nm. With addition to the process a final solution of maltodextrin in 4-6 volumes of its weight in water was added in slow stirring and adjusting the TDS of the solution to 25-27%. The method further includes spray-drying the nano-emulsion with the inlet temperature at 155° C. and the outlet temperature of 95° C. to get a laminar flowing proliposomal formulation in the powder form.

In one embodiment, the formulation is in a powder form. In one embodiment, the formulation is in a liquid form. The formulation may be compressed to prepare tablets.

In certain embodiments, the partially hydrolyzed guar gum stabilizes the liposomal structure by forming a dietary fiber coating, thereby enhancing gastric tolerance and reducing gastric irritation.

In various embodiments, the liposomal vesicles are configured to improve absorption and mitigate oxidative stress, immune function, cardiovascular health, and skin health.

In certain embodiments, the process further includes a clinical validation step to demonstrate superior bioavailability of the proliposomal formulation compared to conventional vitamin C supplements.

While the foregoing description discloses various embodiments of the invention, other and further embodiments of the invention may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of the following examples. However, it is to be understood that the foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Example 1: Preparation of the Proliposomal Formulation

The Liposomal formulation makes use of the lipid bilayer more preferably by lecithin, using sunflower lecithin as the lipid source. Ethanol and sunflower lecithin with a content of phosphatidylcholine 70% more specifically were mixed under controlled stirring at a temperature of 50-55° C. to form the homogeneous lipid mixture as the organic phase. This mixture is then combined with water preferably taking polysorbate 80 in an abiding wt content % of 1-2% as surfactant and stirred vigorously for 20-30 minutes at the same temperature to form a stable homogenous emulsion, which is subsequently cooled to 30-35° C. for vesicle stabilization. The stabilized emulsion is then incorporated with ascorbic acid in defined portions with a slow addition process along with 1.5-2.0 times its weight of water in subsequent additions, to form the homogeneous emulsion after 30-45 minutes of stirring leaving any undissolved ascorbic acid in solution.

To this emulsion, the solution of partially hydrolyzed guar gum (Dietary Fiber) (3.0-5.0% by weight) dissolved in 1.5-2.0 times its weight of water was added, creating a homogenous emulsion stage. The resulting nano-emulsion was processed through a high-pressure homogenizer to achieve liposomal vesicles with diameters ranging from 100 to 200 nm. To further enhance stability and convert the formulation into the proliposomal form, the aqueous solution of plant-based carbohydrates or starch derivatives (preferably aqueous solution of maltodextrin) was added to the nano-emulsion for secondary coating as a carrier and stabilisation during spray drying. The final mixture was spray-dried with the solid content of 25-27%, using the inlet temperature of 150-155° C. and the outlet temperature of 90-95° C. to give a stable powder of proliposomal vitamin C.

### Example 2: Clinical Study

A randomized, open label, two arm, cross-over trial to assess the bioavailability of liposomal and non-liposomal vitamin-C formulations in healthy volunteers is carried out. Capturing demographic data, medical history, physical and systemic examination vitals parameters including respiratory rate, ECG, chest X-ray (PA View), hematology, biochemistry, serology and urine analysis were carried out. Eligible subjects were randomized into two groups:
Product I (treatment A): Liposomal Vitamin C (test product)
Product II (treatment B): Non-Liposomal Vitamin C (control product)
12 subjects were involved in this study 6 for each treatment group.
Dose & Dosage form: Single dose 500 mg (2 Capsules at a time)

In-Housing procedure: Subjects who cleared the inclusion exclusion criteria were checked-in to the clinical facility from at least 12 hrs prior to the Investigational Product administration. Subjects were under supervised feeding during the study period and were housed in the clinical facility until 24 hrs post dose.

Study Product Administration: After overnight fasting for at least 10 hrs, specified dose of the study products were administered through oral route to each subject in sitting posture, at ambient temperature, as a single dose, in study period, as per the randomization code list. Dosing activity was followed by mouth check to assess the compliance to dosing. Total 12 subjects received two (2) products in period I (6 subjects per each arm), the required blood samples were collected. A washout period of 5 days from day 0 was maintained prior to commencing period II. In period II, similar procedure was carried out as in period I, except that the same subjects in each arm will receive a different product in Period II. By end of the study, every subject received the 2 study products in this cross-over design, thus eliminating the inter and intra subject variations in the product absorption.

Blood Sampling Point: In each study Period, total 11 (1×10 ml each) blood samples were collected from each subject and total 11 blood samples in entire study for Pharmacokinetic Analysis. Time points are 0 min, 30 min, 60 min, 90 min, 120 min, 3 hrs, 4 hrs, 6 hrs, 8 hrs, 12 hrs and 24 hrs.

Phlebotomy and Cannulation: On the day of the study, phlebotomy was performed within 01 hours prior to dosing. An indwelling I.V cannula/scalp vein was placed in-situ of a forearm vein of the subjects till 12 hours.

Sample preparation and storage: All the blood samples were placed in wet ice water bath after each sample collection, once blood samples from all the study participants at each time point collected, these were transferred for centrifugation at 5000±100 RPM for 15 mins. After centrifugation plasma was carefully separated into a suitably labelled micro centrifuge tubes using micropipette as individual aliquots and stored in a deep freezer at a temperature-20° C. or colder for interim storage. After every 2 hours, the collected plasma samples were transported to −80° C. deep freezer for storage. At completion of study, all the samples were transported to analytical lab.

Vital signs of the subjects were assessed at admission, prior to dosing (00.00 hrs.) and at are 15 mins, 30 mins, 60 mins, 120 mins, 4 hrs, 8 hrs, 12 hrs post-dose and at the time of checkout (24.00 hours). Physical examination of the subjects was done at admission 1, 2, 4, 8, 12 and 24 hours post dose. Blood and urine samples of the subjects were collected to conduct the safety analysis.

Analytical procedure and statistical analysis: The plasma samples were analyzed using fit-for-purpose LC-MS/HPLC method for the quantification of Vitamin C in samples. The calibration curve (CC) for the method will consist of at least 6 non-zero calibration standards along with a blank and blank with internal standard samples. Study samples were analyzed along with quality control samples (low, medium and high QC samples). The pharmacokinetic parameters were calculated using the standard analytical tool. The relative bioavailability of the test formulations was compared and reported for AUC (0-24 h) from the same analysis with respect to standard Vitamin C formulation. All statistical analyses were performed using SAS version 9.4. Descriptive statistics (N, mean, standard deviation, median, minimum and maximum) were provided for quantitative data. Paired t-test was used for analysis of all parameters change from baseline data compared with test groups treatment. ANCOVA was evaluated for the impact of Test group vs comparator group. Statistical significance was accepted at 5% level of significance (P<0.05).

### Table 1 Shows Vitamin C Concentration in Plasma at Different Time Intervals with Treatment A (ng/ml)

| | | | Std. | | | | |
|---|---|---|---|---|---|---|---|
| Time of | | | Error of | | Std. | | |
| Assessment | | Mean | Mean | Median | Deviation | Minimum | Maximum |
| 0 | hour | 271.98 | 12.90 | 271.32 | 44.68 | 221.52 | 396.56 |
| 0.5 | hour | 1467.50 | 32.45 | 1429.06 | 112.40 | 1310.97 | 1684.55 |
| 1 | hour | 4471.73 | 52.24 | 4465.53 | 180.98 | 4281.92 | 4835.68 |
| 1.5 | hours | 5711.62 | 93.88 | 5540.55 | 325.20 | 5390.91 | 6475.98 |
| 2 | hours | 8687.97 | 364.96 | 8795.21 | 1264.26 | 6715.28 | 10988.96 |
| 3 | hours | 11315.41 | 297.46 | 11758.19 | 1030.42 | 9500.29 | 12737.90 |
| 4 | hours | 16462.86 | 71.15 | 16481.39 | 246.47 | 16111.17 | 16792.90 |
| 6 | hours | 9484.32 | 794.79 | 10279.68 | 2753.25 | 1056.28 | 11388.94 |
| 8 | hours | 6401.10 | 200.82 | 6242.55 | 695.68 | 5476.05 | 7953.77 |
| 12 | hours | 3274.65 | 108.81 | 3262.07 | 376.93 | 2793.51 | 4284.21 |
| 24 | hours | 1674.29 | 56.04 | 1676.53 | 194.12 | 1305.13 | 1953.10 |

### Table 2 Shows Vitamin C Concentration in Plasma at Different Time Intervals with Treatment B (ng/ml)

| | | | Std. | | | | |
|---|---|---|---|---|---|---|---|
| Time of | | | Error of | | Std. | | |
| Assessment | | Mean | Mean | Median | Deviation | Minimum | Maximum |
| 0 | hour | 149.95 | 4.08 | 154.14 | 14.15 | 125.76 | 168.24 |
| 0.5 | hour | 564.60 | 11.48 | 559.30 | 39.76 | 512.20 | 642.14 |
| 1 | hour | 1565.51 | 36.67 | 1574.00 | 127.03 | 1389.63 | 1703.25 |
| 1.5 | hours | 1901.53 | 30.14 | 1891.80 | 104.42 | 1765.12 | 2128.32 |
| 2 | hours | 4427.89 | 65.14 | 4355.30 | 225.64 | 4215.04 | 4852.36 |
| 3 | hours | 5991.78 | 54.34 | 5987.46 | 188.23 | 5578.44 | 6278.41 |
| 4 | hours | 6950.59 | 147.00 | 6886.33 | 509.22 | 6387.14 | 7896.34 |
| 6 | hours | 5343.34 | 77.13 | 5288.56 | 267.18 | 5019.63 | 5986.32 |
| 8 | hours | 2356.80 | 86.45 | 2229.13 | 299.46 | 2013.31 | 2987.43 |
| 12 | hours | 1770.04 | 51.44 | 1781.94 | 178.20 | 1498.32 | 1987.36 |
| 24 | hours | 1162.57 | 57.87 | 1151.26 | 200.46 | 845.03 | 1401.37 |

### Table 3 Shows Paired Comparisons of Treatment Groups of Vitamin C Levels in Plasma by Parametric Test (Independent Samples Test)

Group Statistics

| | | | | Std. | Std. Error |
|---|---|---|---|---|---|
| Parameters | | N | Mean | Deviation | Mean |
| Groups | Treatment A | 12 | 16462.8601 | 246.47362 | 71.15080 |
| | Treatment B | 12 | 6950.5895 | 509.21879 | 146.99880 |

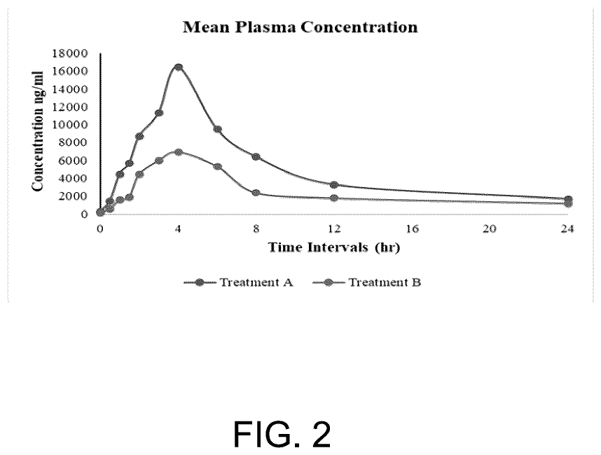

FIG. 2 exhibit mean concentration of plasma vitamin C with different formulations. The relative oral bioavailability of liposomal vitamin C when measured in terms of the mean Cₘₐₓ achieved in the plasma was 2.36 folds higher at 16462.86 ng/ml compared to the non-liposomal vitamin C measured at 6950 ng/ml.

Ingestion of a liposomal Vitamin C supplement differentially affects the concentrations of vitamin C in the blood, volume of distribution, clearance rates, and elimination from the blood compared to a non-liposomal vitamin C supplement. These findings are important because they are the first to demonstrate and influence the nutrient bioavailability. This study evaluated the safety and pharmacokinetic parameters for bioavailability of Liposomal Vitamin C formulation. The study concluded that the study drug Liposomal vitamin C formulation has better superior bioavailability (Cₘₐₓ and AUC) compared to the non-liposomal vitamin C preparation. The study also concludes that Liposomal vitamin C is better bioavailable by 2.36 folds over the non-liposomal vitamin C formulation.

### Advantages of the Present Disclosure

The formulation is a safer and more bioavailable form of vitamin C designed to minimize gastric discomfort.

The formulation stabilizes the liposomal structure and improves the bioavailability of the active ingredient (vitamin C), making the formulation an ideal nutraceutical supplement.

The formulation offers a nutraceutical supplement aimed at managing oxidative stress, enhancing immune system function, supporting heart health, and improving skin.

The encapsulation of ascorbic acid within liposomes facilitates better cellular uptake, ensuring more effective delivery to target tissues.

The formulation protects vitamin C from oxidative degradation which increases shelf life and potency of the supplement, making it suitable for long-term storage and transport under varying environmental conditions.

The formulation is free from chitosan and utilizes a neutral polysaccharide polymer to enhance gastric comfort and prebiotic benefits.

We claim:

1. A proliposomal formulation, comprising:
70 to 75 wt % of ascorbic acid (L-ascorbic acid);
1 to 2 wt % of surfactant;
4 to 5 wt % of sunflower lecithin;
3 to 5 wt % of dietary fibers; and
14 to 20 wt % of a polysaccharide carrier.

2. The proliposomal formulation as claimed in claim 1, wherein the surfactant is selected from a group consisting of polysorbate 80, polysorbate 60, polysorbate 40.

3. The proliposomal formulation as claimed in claim 1, wherein the sunflower lecithin comprises a phosphatidylcholine content ranging from 50 to 70%.

4. The proliposomal formulation as claimed in claim 1, wherein the dietary fibers are selected from a group consisting of partially hydrolyzed guar gum, guar gum, pectin.

5. The proliposomal formulation as claimed in claim 1, wherein the polysaccharide carrier is selected from a group consisting of maltodextrin, gum acacia and a combination thereof.

6. The proliposomal formulation as claimed in claim 1, wherein the proliposomal formulation comprises liposomal vesicles with a diameter ranging from 100 to 200 nm and a solid content of 25 to 27%.

7. A method for preparing a proliposomal formulation, comprising steps of:
a) mixing 4 to 5 wt % of sunflower lecithin with ethanol at a temperature of 50 to 55° C. under controlled stirring to obtain an organic phase;
b) mixing the organic phase of step a) with an aqueous phase containing 1 to 2 wt % of surfactant to form a stable emulsion followed by cooling the stable emulsion to stabilize vesicles;
c) incorporating 70 to 75 wt % of ascorbic acid into the cooled stable emulsion of step b) with continuous stirring for 30 to 45 minutes to form a homogeneous emulsion;
d) adding 3 to 5 wt % of a dietary fiber solution to the homogeneous emulsion of step c) to create a final liposomal emulsion with loaded ascorbic acid;
e) homogenizing the final liposomal emulsion of step d) at pressure in a range of 200-300 bar to achieve vesicle size ranging from 100 to 200 nm for a homogenised nano-emulsion; and
f) spray-drying the homogenized nano-emulsion of step e) with 14 to 20 wt % of a polysaccharide carrier solution to produce a proliposomal formulation.

8. The method as claimed in claim 7, wherein the mixing at step b) is carried out by stirring vigorously for a duration of 20 to 30 minutes at 50 to 55° C.; wherein the stable emulsion is cooled at step b) at a temperature ranging from 30 to 35° C. to stabilize vesicles.

9. The method as claimed in claim 7, wherein the spray-drying at step f) is carried out at an inlet temperature of 150 to 155° C. and an outlet temperature of 90 to 95° C.

10. The method as claimed in claim 7, wherein the surfactant is selected from a group consisting of polysorbate 80, polysorbate 60, polysorbate 40; wherein the dietary fibers are selected from a group consisting of partially hydrolyzed guar gum, guar gum, pectin; wherein the polysaccharide carrier is selected from a group consisting of maltodextrin, gum acacia and a combination thereof.
